(19) United States
(12) Patent Application Publication
(10) Pub. No.: US 2013/0225924 A1
(43) Pub. Date: Aug. 29, 2013

(54) MEDICAL SCOPE WITH SINGLE USE PROBE

(71) Applicants: Norman Simms, Manahawkin, NJ (US); John Lescota, Middlesex, NJ (US)

(72) Inventors: Norman Simms, Manahawkin, NJ (US); John Lescota, Middlesex, NJ (US)

(21) Appl. No.: 13/883,298

(22) PCT Filed: Nov. 2, 2011
(86) PCT No.: PCT/US11/01846

§ 371 Date: May 3, 2013

Related U.S. Application Data
(60) Provisional application No. 61/456,684, filed on Nov. 10, 2010.

(51) Int. Cl. A61B 1/00; A61B 1/06; A61B 1/005; A61B 1/05

(52) U.S. Cl. CPC A61B 1/00062; A61B 1/00103; A61B 1/05; A61B 1/00045; A61B 1/06; A61B 1/0058; A61B 1/00126
USPC 600/109; 600/118

(57) ABSTRACT

A medical scope with a probe capable of being utilized for a single imaging procedure is described. The medical scope has a control unit with a programmable memory unit, a socket for receiving a plug end of the probe, and the probe having a light guide and a pair of power wires extending along the length thereof. The control unit also has a display screen, a set of switches to control functions, and a light source. The probe includes a check bit memory unit for retaining a selected value that the control unit will detect, a zero value indicating a new probe and a non-zero value indicating a used probe. Therefore, the used probe will be rejected by the control unit and cannot be used again.

### RELATED APPLICATION

This application is a national phase of PCT/US2011/001846, filed on Nov. 2, 2011 which is a conversion of U.S. provisional patent application No. 61/456,684, filed on Nov. 10, 2010.

### FIELD OF THE INVENTION

The present invention relates to the field of medical imaging devices, and more particularly to a medical scope with a probe for obtaining an image within an enclosed body space and with a detection protocol to ensure that the probe is only used once.

### BACKGROUND OF THE INVENTION

Medical science conducts many diagnostic procedures with devices utilizing a vision enabled probe. The probe is inserted into a body opening, e.g. the esophagus, in order to obtain images within specific body areas to enable the medical professional to reach a diagnosis and develop a treatment regimen. The probe typically includes a source of illumination and means for conducting the image to the viewing physician or a recording medium.

A major problem encountered in many hospitals and clinics is the spread of infection from a first patient to a second patient. In the case of devices using a probe to acquire an image, the probe is in intimate contact with the first patient and is likely to pick up undesirable microorganisms. To minimize the chance of cross contamination by using the probe on a second patient, the currently known probes must be sterilized after each use. Sterilization is a time consuming and expensive process. In addition, there is no certain way to know that a probe has been thoroughly sterilized or has not been exposed to infectious contamination after sterilization. For these reasons, a probe may be inadvertently used without being sterile, endangering the next patient being examined with the probe.

### SUMMARY OF THE INVENTION

The present invention provides an apparatus and method that avoids the problems described above by utilizing a probe that is used only once and is discarded after use. The probe incorporates an illumination source and a camera. The apparatus has an microprocessor control unit and a probe that has been initialized to a selected value. The probe is connected to the control unit and the control unit reads the selected value. If the selected value read from the probe agrees with a selected value allowed by the control unit, the control unit accepts the probe. The control unit then assigns an identification to the probe that is not equal to the accepted selected value. The control unit next energizes the probe to actuate the illumination device and the camera to enable the medical professional to conduct the patient examination. Upon completion of the examination and retraction of the probe, the probe is separated from the control unit and discarded. Nevertheless, if an attempt is made to use the already used probe, the control unit verification of the selected value would fail and the probe would not function.

### BRIEF DESCRIPTION OF THE DRAWINGS

The present invention is best understood in conjunction with the accompanying drawing figures in which like elements are identified by similar reference numerals and wherein:

FIG. 1 is a perspective view of a control unit for the medical scope of the present invention.

FIG. 1A is a view of the internal connective surface of a socket in the control unit.

FIG. 2 is a perspective view of a single use probe for use with the control unit of FIG. 1.

FIG. 3 is an enlarged perspective view of the end portion of the probe of FIG. 2.

FIG. 4 is a flow chart of the operation of the medical scope with single use probe according to the present invention.

### DETAILED DESCRIPTION OF THE PREFERRED EMBODIMENT

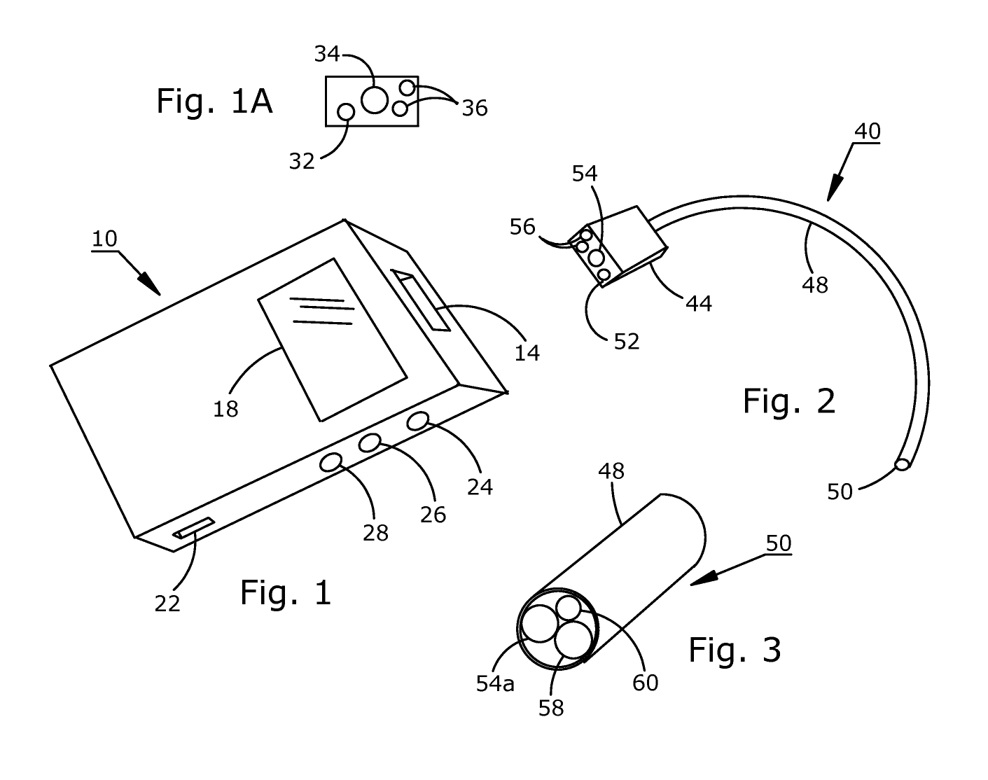

Referring to FIG. 1, a microprocessor control unit 10 is illustrated in perspective view. Control unit 10 has a socket 14 adapted for receiving a probe plug and activating the operative components of a probe assembled to the plug. Control unit 10 incorporates a programmable memory unit (PMU) to interact with display screen 18 and other components of the invention. A display screen 18 is incorporated in control unit 10. A series of switches 24, 26 and 28 are connected to the operating circuitry of control unit 10 for actuating control unit 10 and the probe, adjusting the brightness of a light to be transmitted within the probe, and inverting an image on display 18 by 180° for better image analysis. It is noted that control unit 10 and probe actuating switch, i.e. an “on” switch, has no “off” function. Once control unit 10 and the probe are actuated, the circuitry remains active until the probe is removed from socket 14. Switches 24, 26 and 28 are shown as examples, and may be used to perform other functions or additional switches added according to the intent of the manufacturer. The sequence of the switch functions is optional at the discretion of the manufacturer. Similarly, operation of control unit 10 through touch screen switch positions on display screen 28 is considered within the scope of the present invention. An output port 22 such as a USB socket is provided in control unit 10 to enable images seen on display screen 18 to be transmitted to an external device, e.g. a printer or a memory cache.

Referring now to FIG. 1A, a view inside socket 14 (FIG. 1) is shown to illustrate the connective components therein. A check bit contact 32 is provided to engage a check bit with the PMU to determine an imbedded value. A light source 34 is positioned to project light through a light guide. A pair of electric contacts 36 are adapted for energizing and communicating with a camera within the probe to be described below.

Referring now to FIG. 2, a probe 40 is seen in a position for being engaged with socket 14 of control unit 10 (FIG. 1). Probe 40 has a plug 44 at a first end, a flexible shaft 48, with a tip 50 at the distal end thereof. Plug 44 is sized to engage socket 14 of control unit 10 (FIG. 1). A check bit memory unit (not visible) is embedded within plug 44. The check bit is capable of being programmed with a selected value and retaining that value until the check bit is re-programmed with a new value. A check bit terminal contact 52 is exposed at the connecting surface of plug 44 to allow the control unit 10 (FIG. 1) to verify the condition of probe 40 by determining the value saved in the check bit. Plug 44 also has a light guide receptor end 54 positioned to connect to a light source that resides within socket 14 of control unit 10. An optic fiber extends from light guide receptor end 54 to probe tip 50 within flexible shaft 48. By locating the light source in control unit 10, tip 50 of probe 40 does not become as hot as it would if a light source were located at tip 50. A pair of power contacts 56 connect to an electric power source, e.g. a battery, within socket 14. For certain diagnostic procedures, it is desirable that shaft 48 is semi-rigid, for which purpose a conformable semi-rigid member, e.g. a stainless steel wire, is incorporated into shaft 48.

Referring now to FIG. 3, an enlarged perspective view of the end portion of shaft 48 is illustrated. As shown, tip 50 includes a light guide emitter end 54a and a lens 58 of a subminiature camera. Light guide emitter end 54a is connected to light guide receptor end 54 (FIG. 2) by a continuous fiber optic light guide within shaft 48. Power contacts 56 (FIG. 2) are connected to supply electric power to and transmit digital signals from the camera behind lens 58. A conformable member 60 in the form of a malleable stainless steel wire is incorporated to enable shaft 48 to be bent into a desired shape to facilitate certain diagnostic procedures. Conformable member 60 terminates within shaft 48 and is not seen at the connecting end of plug 44.

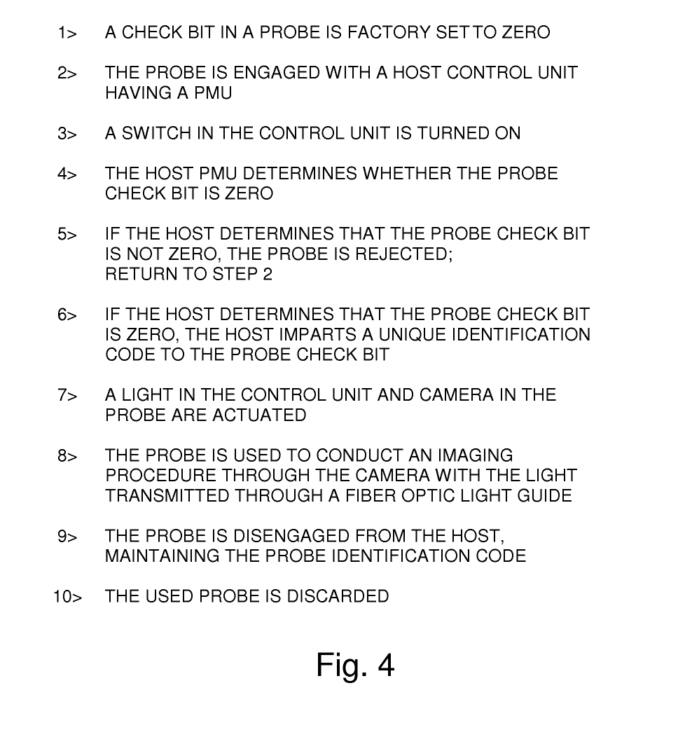

Referring now to FIG. 4, a flow chart is provided to describe the operational steps of the present invention. Control unit 10 incorporates a programmable memory unit (PMU) to interact with display screen 18, switches 24, 26 and 28 (FIG. 1) and with probe 40 (FIG. 2). Probe 40 incorporates a check bit that may be electromagnetically actuated to carry a small amount of information, e.g. an identification number. According to step 1, the check bit in each probe is set to a selected value during manufacture of zero, i.e. blank or empty. Alternate values for the check bit initialization may be selected. In step 2, the probe is physically engaged with the host control unit and the built in PMU. In step 3, the control unit switch is turned on, and in step 4 the host PMU determines whether the probe check bit is equal to the selected value. In step 5, if the MCU finds the check bit in the probe is at a value other than the selected value, the probe is rejected and the program returns to step 2 to engage and qualify a different probe. In other words, if the probe being connected to the control unit has been used, indicated by a non-zero check bit reading, the used probe cannot be activated and must be discarded. In step 6, if the PMU determines that the check bit of the probe is zero, the PMU overrides the selected value and attaches a unique identification code into the check bit memory. Next, in step 7, the PMU turns the light source on and connects power to the camera. The display indicates in step 8 that the probe is ready, allowing the diagnostic procedure to be conducted. The diagnostic procedure is performed by inserting the shaft of the probe into a body orifice, generating image information to the display on the control unit. In step 9, at the conclusion of the diagnostic procedure, the probe is disengaged from the control unit host, and in step 10 the used probe is discarded.

To reiterate, according to the flow chart of FIG. 4 described above, once a probe has been used, the check bit has been reset from an initial selected value of zero to carry a unique identification code. The control unit PMU will not accept as new (and sterile) a probe with a non-zero reading, thus a used probe cannot be used again and must be discarded. It is further noted that since the invention prevents a probe from being used more than once, a new probe must be supplied for each successive procedure.

According to the preferred embodiment of the invention, the probe external parts are formed of a medical grade silicone resin, for example a Dow Corning No. 935 silicone. In practice, the probe flexible shaft is made in varying diameters, including a pediatric version having an outside diameter of 4.6 mm.

While the description above discloses preferred embodiment of the present invention, it is contemplated that numerous variations and modifications of the invention are possible and are considered to be within the scope of the claims that follow.

What is claimed is:

1. A medical scope with a single use probe, comprising:
a. a control unit having a socket formed therein;
b. a probe having a flexible shaft with a plug mounted at one end thereof, the plug adapted for engaging the socket;
c. wherein the probe comprises a check bit capable of retaining a selected value; and
d. the control unit being operational to detect a value in the probe and accept only a probe having a value equal to the selected value.

2. The medical scope with a single use probe described in claim 1, wherein the selected value is initially set to zero.

3. The medical scope with a single use probe described in claim 1, wherein the control unit further comprises a display screen.

4. The medical scope with a single use probe described in claim 1, further comprising a camera mounted at a distal end of the probe shaft.

5. The medical scope with a single use probe described in claim 1, wherein the control unit comprises a switch for energizing the control unit and the probe, and wherein the energized control unit is de-energized only by disengaging the probe plug from the control unit socket.

6. The medical scope with a single use probe described in claim 1, further comprising a light source residing within the control unit, the light source positioned to project a light through a light guide extending from the probe plug to a distal tip of the probe shaft.

7. The medical scope with a single use probe described in claim, further comprising a conformable semi-rigid member 60 extending through the shaft from the probe plug to a distal tip of the probe shaft.

8. The medical scope with a single use probe described in claim 1, wherein the external parts of the probe are formed of a medical grade resin.
